(19) United States
(12) Patent Application Publication
(10) Pub. No.: US 2012/0197224 A1
(43) Pub. Date: Aug. 2, 2012

(54) GARMENT WITH MOISTURE TRIGGERED ALARM

(75) Inventor: Avtar S. Chagger, Coventry (GB)

(73) Assignees: Babyweecare Limited, Kent (GB); Avtar S. Chagger, Coventry (GB)

(21) Appl. No.: 13/395,167

(22) PCT Filed: Sep. 13, 2010
(86) PCT No.: PCT/GB2010/001721

§ 371 Date: Apr. 4, 2012

(30) Foreign Application Priority Data
Sep. 11, 2009 (GB) 0915909.6

(51) Int. Cl. A61F 13/42

(52) U.S. Cl. 604/361

(57) ABSTRACT

A garment for incontinent children or adults is provided. The garment comprises a detection unit that has a moisture sensor for sensing excreta or bodily fluids, a response means or producing a response signal in response to excreta being sensed by the sensor means; and a transmitter means for transmitting a trigger signal in response to the response signal. The garment also comprises a receiver unit having a receiver for receiving said trigger signal and alert means for generating an alert signal in response to receipt of said trigger signal.

The present invention relates to an alarm for incontinent children or adults.

Undergarments for children or adults having faecal or urinary incontinence are well known. Such garments include, for example, nappies, diapers, toddler training pants, incontinence pants etc. It is also known for disposable nappies or training pants to include external decorative images, which disappear in the presence of moisture, thus alerting a parent or other carer that the undergarment is wet.

Such images are of limited benefit, however, when a child is wearing other external garments over the top of a nappy or training pants. Such external garments obscure the decorative images thus preventing the carer from being able to easily determine the status of the undergarment. This also means that there is inevitably a delay in the carer becoming aware that the undergarment is wet. Such delay leads to discomfort for the wearer and increases the potential for moisture related skin disorders.

The present invention provides an improved garment for incontinent children or adults that overcomes or at least mitigates the above problems.

Accordingly, the present invention provides a garment for incontinent children or adults comprising: a detection unit having: a moisture sensor for sensing excreta or bodily fluids; response means for producing a response signal in response to excreta being sensed by the sensor means; and transmitter means for transmitting a trigger signal in response to the response signal; a receiver unit having: a receiver for receiving said trigger signal; and alert means for generating an alert signal in response to receipt of said trigger signal.

The present invention will now be described, by way of example only, with reference to the figures in which:

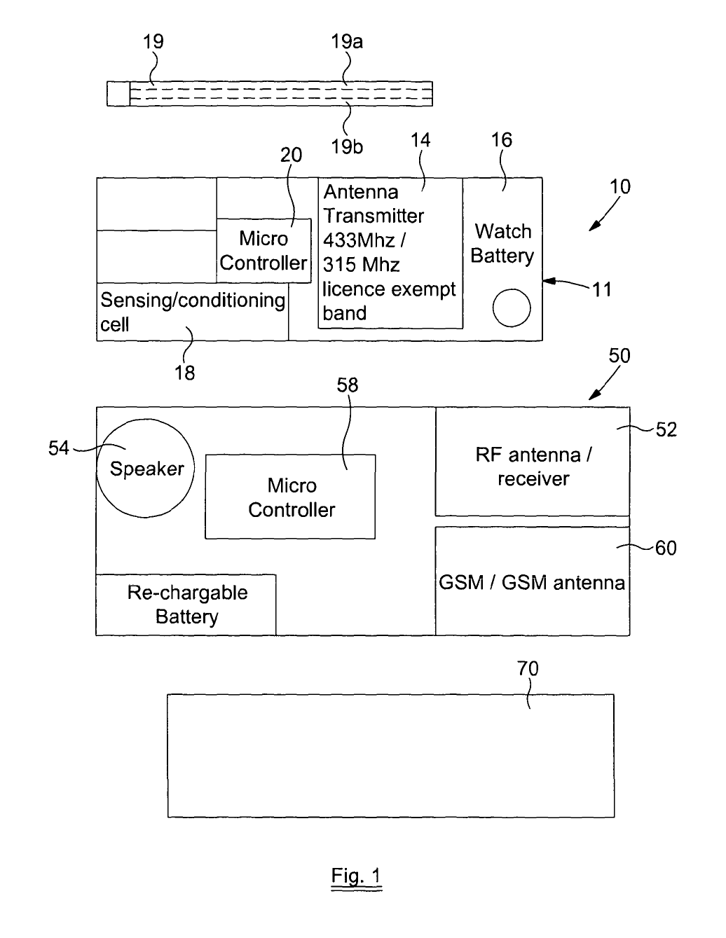

FIG. 1 shows a block diagram of a system for excreta detection;

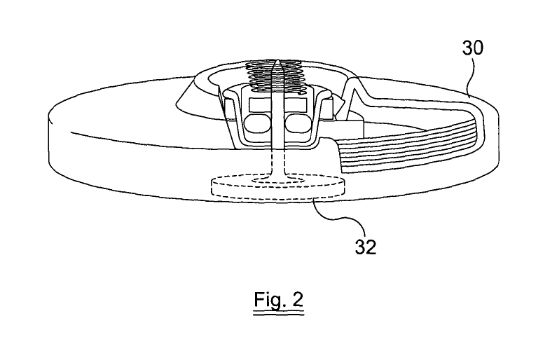

FIG. 2 shows a preferred form of detection unit of the system of FIG. 1.

The preferred system is described below in relation to a diaper but may equally be used in other circumstances and garments where its function would be desirable.

In FIG. 1 a first embodiment of a system for excreta detection is shown generally at 10. The system 10 comprises a detection unit 11, and a receiver unit 50. The detection unit 11 has a transmitter 14, a battery 16, a moisture sensor 18 and a microcontroller 20 for controlling operation of the detection unit.

The sensor 18 has a sensor strip 19 which comprises first and second proximate sensor electrodes 19a, 19b. The first and second electrodes 19a, 19b are spaced at a predetermined distance from one another. The predetermined distance is set according to its suitability for sensing a change in resistance or potential difference arising from the presence of moisture arising from excreta such as uric acid. The first sensor electrode 19a is aluminium and the second sensor electrode 19b is copper. It will be appreciated, however, that the electrodes may be made of any suitable material and that both electrodes may be made of the same material.

The sensor electrodes 19a, 19b are implemented in the form of two conducting tracks printed onto a suitable substrate for incorporation into the garment. The substrate is flexible for the comfort of a person wearing the garment in use. It will be appreciated, however, that the sensor electrodes 19a, 19b may be implemented in any suitable manner. For example, the sensor electrodes 19a, 19b may be in the form of conductors in the form of bare wires, or other conductors sown either directly into the fabric of the garment or into a patch for incorporation in the garment. Alternatively, the sensor electrodes 19a, 19b may be implemented as two conductors spaced from one another by a strip of suitable insulating material of appropriate thickness. Additionally, the sensor electrodes 19a, 19b may be arranged in a spiral formation for improved compactness and reliability. The detection unit 11 and sensor strip 19 may be incorporated into a garment such as a baby's diaper, for example by slotting into a small pocket in the diaper. Alternatively, the sensor strip 19 may be printed onto the diaper. Typically, the sensor strip 19 is constructed in a similar manner to blood sugar type sensors and clips into a suitable receptor in the sensor 18 which electrically connects with the microcontroller 20. The microcontroller 20 monitors a change in resistance or potential difference at the sensor arising from the presence of moisture from excreta such as uric acid.

The microcontroller 20 produces a detection signal in response to excreta being sensed by the sensor 18. Typically the microcontroller compares the signal with a predetermined threshold.

The sensor provides constant monitoring of the moisture within a diaper. If the resistance between the sensor strip electrodes rises above a preset level then the microcontroller 20 generates a signal for transmitting to the receiver unit 50.

The receiver unit 50 has a receiver 52, speaker 54, battery 56, microcontroller 58 and antenna 60. On receipt of the signal transmitted by the detection unit 11 the microcontroller 58 generates an alert signal which can be used to generate a visual and/or an audible alarm. Any suitable electronic sound producing device may be used in place of the speaker 54 for producing sound in response to the alert signal. Alternatively, the sound producing device may be in the form of a piezo-electric buzzer driven by the alert signal.

The receiver unit 50 is powered by the battery 56.

The receiver unit 50 is in the form of a communications device configured to transmit an alert signal to the speaker 54 and/or an external device in response to a detection signal produced by the microcontroller 58. The communications device may be of any suitable form for example a radio, SMS, or Bluetooth enabled transmitter. The alert signal is configured for receipt by a suitable external device such as a mobile phone, laptop, computer, dedicated hand held device or the like 70 to alert a carer that the garment is wet.

A typical use of the first embodiment will now be described by way of example.

In operation, when the baby urinates into the diaper the moisture from the urine spreads and lowers the resistance of the region between the sensor electrodes 19a, 19b. The microcontroller 20 detects this change in resistance and compares it with a reference level to produce a corresponding detection signal in response. The detection signal is then transmitted to the receiver unit 50 which drives the speaker 54 to produce an audible warning to alert a carer that the diaper is wet. The carer then removes the sensor 18 thus discontinuing the alert. The carer can then replace the wet diaper with a dry diaper.

FIG. 2 shows a practical form of the detection unit 11. Here, the unit 11 has a main body 30 which houses the transmitter 14, battery 16 and microcontroller 20 and moisture sensor 12. The detection unit also has a pin means 32 which is used to secure the detection unit 11 to the diaper in a manner similar conveniently to that used for anti-theft garment tags. In this embodiment, the pin 32 conveniently serves as the sensor strip having the moisture sensor electrodes 19a, 19b which run parallel along the pin and engage with suitable sensor 18 contacts in the main body 30. The electrodes are exposed at the pin head to sense moisture.

It will be appreciated that other embodiments are possible incorporating a combination of the features described and/or additional features or alternatives. In particular the invention is not limited to detecting excreta but also applies to other bodily fluids such as blood.

The sensor, may be configured to sense solid as well as wet excreta using other parameters. For example, the sensor 18 may include temperature detection means for sensing a temperature change indicative of the presence urinary and/or faecal excreta. The temperature detection means may comprise, for example, sensor electrodes incorporating a bi-metallic strip, semiconductor temperature sensor, or similar, which produce a change in resistance in the event of a temperature change. The change in resistance may be a change from an open to a short-circuit or vice versa.

The sensor 18 may also include sensor electrodes configured to produce an electric current or potential difference as a result of an electrolytic reaction in the presence of uric acid.

The sensor 18 may also comprise a weight, mass or pressure switch configured to operate when the mass of excreta in the diaper exceeds a predetermined level and the microcontroller 20 may be configured to produce a detection signal in response to operation of the switch.

The sensor 11 may also include means for detecting chemical characteristics of the excreta or bodily fluids, such as acidity, protein levels or blood sugar levels. Correspondingly, the response means may include means for analysing the output of the sensor, and for producing a response signal dependent on the analysis to alert carers to possible health issues such as diabetes or the like.

The receiver unit 50 may be configured to produce other types of alert, for example, a complex audible alert in the form of music, a voice or the sound of a baby. For example, the music may be in the form of a lullaby or the like. The voice may provide a simple message to indicate, for example, who's turn it is to change a nappy.

The receiver unit 50 may include visual means for producing a visual alert. The visual means may include, for example, a light source such as a light emitting diode or the like.

The receiver unit 50 may be operable to transmit an alert signal in the form of ultrasound or infrared to an appropriate device.

The receiver unit 50 may also be provided with an address for identification purposes, and may be operable to transmit the address as part of the alert signal or separately to allow a carer to identify which diaper has been affected.

If the detection unit 50 is to be used to transmit signals to, for example, a cell phone using SMS technology it first needs to be programmed with the relevant cell phone number. The recipient SMS number used for all outgoing warning messages can be automatically programmed using a simple SMS message in the following format.

A text message is sent to the unit 50 in the following manner:
#S (user's phone number) S
e.g Composing the following message #S07866581586S will cause all alert messages to be sent to GSM number: 07866581586.

If the user wishes to know the status of the unit 50 then he sends the following message to the unit 50:
?S

Unit 50 responds with the following:
Serial No.: Unit 50 04604
Signal Strength: 12 (where 9 is low and 31 is maximum)
Battery Voltage: 3.9v (where 3.3 is low and 4.2 is maximum)
Cell i.d. EECC (current base station cell being used)

An automatic low battery warning can be sent from the detection unit 50 once the internal battery reaches about 3.5 volts. In this case the standard header message:

“Please change my nappy” will be replaced with “LOW BATTERY”

The transmitter 14 is 433 MHz and 315 MHz licence exempt band and the receiver 52 is conveniently Quad Band on 850 MHz, 900 MHz, 1800 MHz or 1900 MHz.

1. A garment for incontinent children or adults comprising:
a detection unit having:
a moisture sensor for sensing excreta or bodily fluids;
response means for producing a response signal in response to excreta being sensed by the sensor means; and
transmitter means transmitting a trigger signal in response to the response signal;
a receiver unit having:
a receiver for receiving said trigger signal;
and alert means for generating an alert signal in response to receipt of said trigger signal.

2. A garment as claimed in claim 1 wherein the sensor means are embedded in the garment.

3. A garment as claimed in claim 1 wherein the garment includes a device pocket for removably receiving the detection unit.
